(19) United States
(12) Patent Application Publication
(10) Pub. No.: US 2012/0302865 A1
(43) Pub. Date: Nov. 29, 2012

(54) SUBJECT INFORMATION ACQUIRING APPARATUS

(75) Inventors: Toshinobu Tokita, Kyoto-shi (JP); Masakazu Taku, Machida-shi (JP)

(73) Assignee: CANON KABUSHIKI KAISHA, Tokyo (JP)

(21) Appl. No.: 13/577,079

(22) PCT Filed: Apr. 19, 2011
(86) PCT No.: PCT/JP2011/002287

§ 371 Date: Aug. 3, 2012

(30) Foreign Application Priority Data
Apr. 21, 2010 (JP) 2010-098024

(51) Int. Cl. A61B 6/00

(52) U.S. Cl. 600/407

(57) ABSTRACT

A subject information acquiring apparatus including a cover which intercepts light, two plates which hold a subject, a laser light source, and a probe which receives an acoustic wave generated from the subject to which the light is emitted from the laser light source, wherein the plates, the laser light source, and the probe are covered by the cover, wherein the cover has a first aperture for inserting the subject as part of a subject person into the cover, and a second aperture which can be opened and closed so that an operator can perform a procedure by inserting a hand of the operator into the cover in the position in which the plane extending each of the plates crosses the cover, is used.

### TECHNICAL FIELD

The present invention relates to a subject information acquiring apparatus which images an acoustic wave emitted from a subject.

### BACKGROUND ART

As a method for specifically imaging angiogenesis caused due to cancer, attention has been drawn to Photoacoustic tomography (PAT). The PAT is a method for emitting light (e.g., near-infrared ray) onto a living body to receive and image an acoustic wave emitted from the inside of the living body by an ultrasound probe. The acoustic wave is also called a photoacoustic wave, and is typically an ultrasound wave. Non Patent Literature 1 (NPL 1) describes a method for acquiring living body information in a living body using the principle of the PAT. Photoacoustic mammography (PAM) in which a subject is a breast is described herein.

FIG. 5 schematically shows the measuring method of NPL 1 in side view. In FIG. 5, an Nd:YAG laser 109 is a light source which emits light to irradiate a subject (breast). An optical system 110 irradiates the subject with the laser light emitted from the Nd:YAG laser 109. A scanning mechanism 111 is equipped with part of the optical system 110, and allows the optical system 110 to perform scanning up or down (in the arrow direction of FIG. 5) and to the right and left (in the depth direction of FIG. 5). An ultrasound probe 112 receives a photoacoustic wave emitted from the subject. The receiving element of the ultrasound probe 112 for imaging is selected from the irradiation position of the scanning mechanism 111, and then, a receiving signal is amplified and is converted to a digital signal for image reconstruction. In addition, a glass window 107 transmits the irradiation light from the optical system 110 therethrough, and presses and holds the subject between the glass window 107 and the ultrasound probe 112. The subject person lies on her stomach on a bed, not shown, and inserts her breast as a subject into the insertion hole of the bed, whereby the breast is pressed and held by the ultrasound probe 112 and the glass window 107. In NPL 1, an image in the living body is acquired from a PAT signal by the method shown in FIG. 5.

### CITATION LIST

### Non Patent Literature

[NPL 1]
Srirang Manohar, et al., The Twente photoacoustic mammoscope: system overview and performance, Physics in Medicine and Biology 50 (2005) 2543-2557.

### SUMMARY OF INVENTION

### Technical Problem

In the above technique, high-output Nd:YAG laser 109 is used as a light source. Therefore, to secure the safety of a subject person and an operator, the entire apparatus is required to be light-shielded. However, NPL 1 does not describe light shielding, and the entire apparatus cannot be sufficiently light-shielded.

In addition, in the PAM, to generate a photoacoustic wave, the light is required to irradiate the region to generate a photoacoustic wave. However, the irradiation light is diffused when entering from the surface of the subject, and is attenuated in the inside of the subject. Therefore, the light cannot always enter deep into the subject. Then, the living body information in the region that the irradiation light cannot reach cannot be acquired. In addition, when the irradiation light reaches there at intensity weakened due to diffusion and attenuation, the sound pressure of the generated photoacoustic wave is lowered to reduce the contrast resolution.

In order that the irradiation light can reach deep in the subject at necessary intensity, the subject is required to be pressed and held so as to reduce the thickness of the subject. For this reason, as when the breast is pressed and held in X-ray mammography, a method by the procedure of the operator (measuring person such as a doctor and a technician) is preferably applied. The procedure is referred to as an operation performed by the operator in such a manner that the operator directly touches the subject, that is, presses and holds the subject by pulling or pressingly extending the subject by hand.

However, NPL 1 does not describe the method for pressing and holding the subject between the ultrasound probe 112 and the glass window 107. In particular, even when the naturally hanged subject is tried to be pressed and held, the subject is moved upward and cannot be sufficiently pressed and held.

The present invention has been made in view of the above problems and an object of the present invention is to provide a technique in which in PAM, the operator can easily perform a procedure when pressing and holding a subject while the safety of the subject person and the operator from irradiation light is secured.

### Solution to Problem

This invention provides a subject information acquiring apparatus comprising:

a cover which intercepts light;

two plates which hold a subject;

a probe which receives an acoustic wave generated from the subject to which the light is emitted from a laser light source; and

a cover which covers the plates and the probe and prevents the light is emitted from the laser light source form emitting to the outside of the apparatus,

wherein the cover has a first aperture for inserting the subject as part of a subject person into the cover, and a second aperture which can be opened and closed so that an operator can perform a procedure by inserting a hand of the operator into the cover in the position in which a plane extending each of the plates crosses the cover.

### Advantageous Effects of Invention

According to the present invention, in PAM, the operator can easily perform a procedure when pressing and holding a subject while the safety of the subject person and the operator from irradiation light is secured.

Further features of the present invention will become apparent from the following description of exemplary embodiments with reference to the attached drawings.

### BRIEF DESCRIPTION OF DRAWINGS

FIG. 1A is a diagram of assistance in explaining the appearance of an apparatus according to Example 1.

FIG. 1B is a diagram of assistance in explaining the inner configuration of the apparatus according to Example 1.

FIG. 1C is a diagram of assistance in explaining another example of the appearance of the apparatus according to Example 1.

FIG. 2 is a diagram of assistance in explaining the inner configuration of the apparatus according to Example 2.

FIG. 3 is a diagram of assistance in explaining the inner configuration of light emission control of the apparatus according to Example 3.

FIG. 4A is a diagram of assistance in explaining the configuration of a procedure door of the apparatus according to Example 4.

FIG. 4B is another diagram of assistance in explaining the configuration of a procedure door of the apparatus according to Example 4.

FIG. 5 is a diagram of assistance in explaining the configuration of an apparatus according to background art.

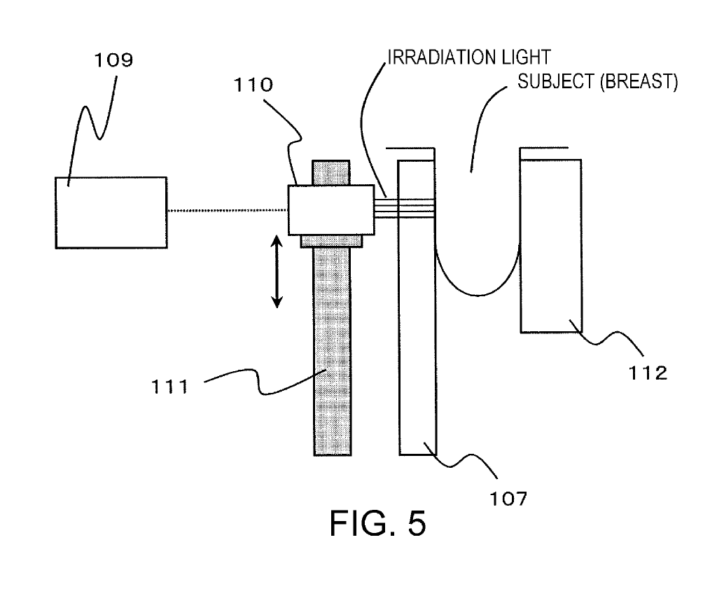

### DESCRIPTION OF EMBODIMENTS

Hereinafter, preferred embodiments of the present invention will be described with reference to the drawings. A subject information acquiring apparatus according to the following examples is a PAM apparatus for acquiring the image of the inside of the breast of the subject person as a subject. Therefore, a photoacoustic wave (e.g., ultrasound wave) emitted from the subject which absorbs the light energy of light from alight source is detected for image reconstruction. It should be noted that the dimensions, materials, shapes, and relative arrangements of components described below should be appropriately changed according to the configuration of the apparatus to which the invention is applied and various conditions and are not intended to limit the scope of the invention to the following description. Further, subject information shows the generation source distribution of a photoacoustic wave generated by light irradiation, the initial sound pressure distribution in the subject (in particular, living body), the light absorption density distribution derived therefrom, and the density distribution of a substance constituting subject tissue acquired from the information. For instance, the substance density distribution shows oxygen saturation.

### Example 1

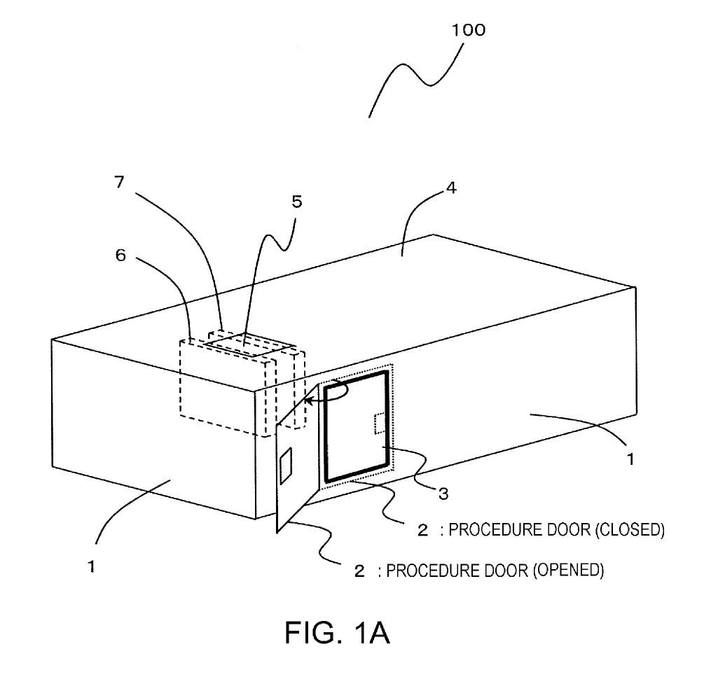

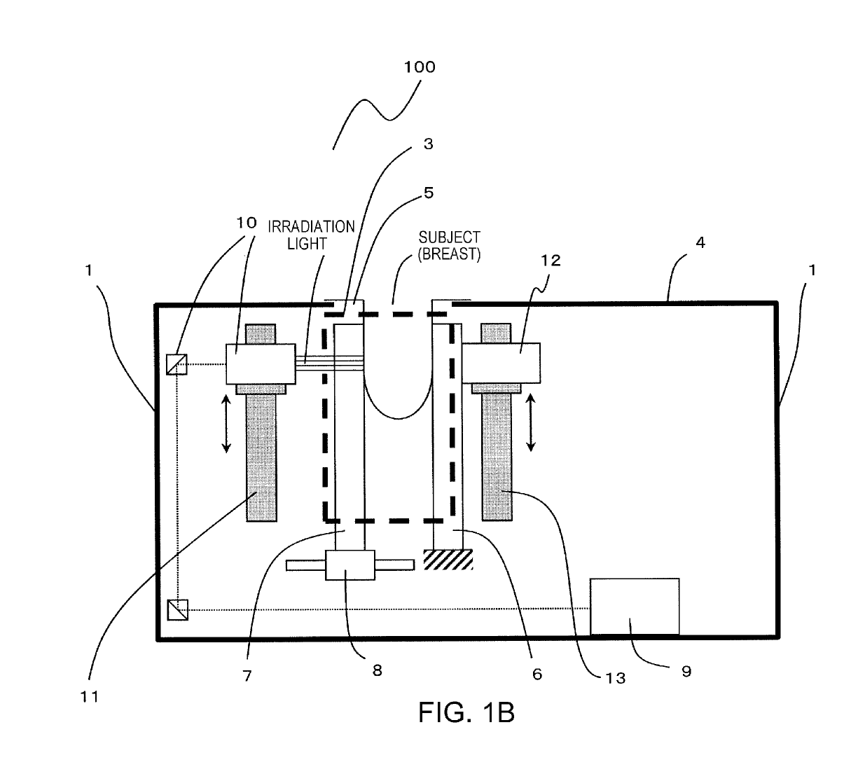

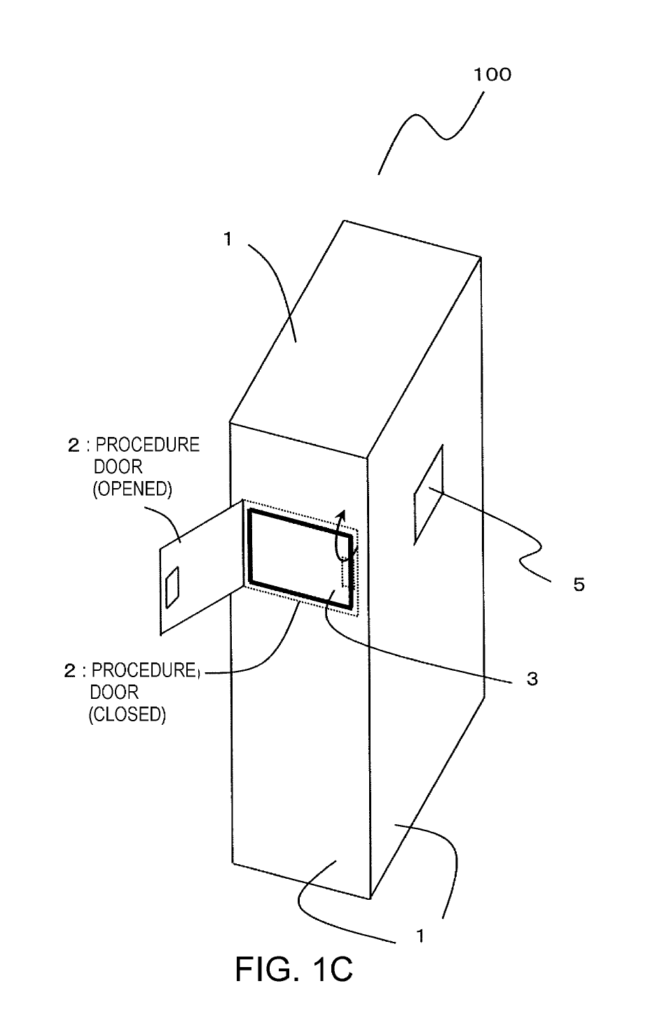

FIG. 1A is a perspective schematic diagram showing the appearance of a PAM apparatus 100 of the present example. The PAM apparatus 100 is covered by an outer cover 1. The outer cover 1 is provided to prevent the light is emitted from the laser light source form emitting to the outside of the apparatus, and seals the PAM apparatus 100.

Part of the outer cover 1 has a procedure door 2. Here, the state that the procedure door 2 is opened is indicated by the solid line, and the state that the procedure door 2 is closed is indicated by the dotted line. Further, in this drawing, a hinged open/close door is shown as the procedure door 2. The procedure door 2 is not limited to this, and may be, e.g., a slide door. When the procedure door 2 is opened, there is a procedure door aperture 3. The subject person, not shown, lies on her stomach on a bed 4, and inserts the subject (breast) from a subject insertion hole 5. Further, the bed 4 is included in part of the outer cover 1. The operator (measuring person such as the doctor and the technician) can insert a hand of the operator from the procedure door aperture 3 into the PAM apparatus to perform a procedure for pressing and holding the subject inserted into the subject insertion hole 5. The subject insertion hole corresponds to a first aperture of the present invention. The procedure door and its aperture correspond to a second aperture.

Then, the configuration of the inside of the PAM apparatus will be described with reference to FIG. 1B. This drawing is a diagram schematically showing the inner configuration of the PAM apparatus 100. A laser light source 9 is a light source for generating a photoacoustic wave, and an Nd:YAG laser and a combination of the Nd:YAG laser and a Ti:sa laser are applicable. An alexandrite laser and an OPO are also effective. An optical system 10 is an optical system which irradiates the subject with the laser light from the laser light source 9 through a pressing plate. An optical scanning mechanism 11 allows the optical system 10 to perform scanning in order to irradiate the large region of the subject with the light.

A probe 12 receives the photoacoustic wave generated from the subject, and has a receiving surface contacted with a holding plate 6 via an acoustic matching material (not shown). The probe 12 is mounted on a probe scanning mechanism 13, and is positioning-controlled with the optical scanning mechanism 11 so that its center is opposite the center of the irradiation light. The photoacoustic wave received by the probe 12 is converted to an electric signal, and is transmitted to a signal processing device, not shown. The signal processing device is a CPU, and amplifies the electric signal to convert the signal to a digital signal for image reconstruction, thereby acquiring the subject information as image data.

Further, although the configuration in which the probe 12 is mounted on the probe scanning mechanism 13 has been described here, the actual configuration is not limited to this. For instance, when a large probe serves as the holding plate 6, the probe scanning mechanism 13 may be omitted. In addition, although in this drawing, the light is emitted from the direction of a pressing plate 7 to the subject and the probe 12 is provided on the holding plate 6, the opposite arrangement is also effective. However, in that case, the pressing plate 7 and the probe 12 are required to be contacted with each other via the acoustic matching material. Alternatively, the optical system and the probe can be arranged on the same plate. Further, the pressing mechanism is provided only on the pressing plate 7. A pressing mechanism 8 may also be provided on the pressing plate 6 and may be pressingly operated. Also in this case, the holding plate 6 and the probe 12 are preferably contacted with each other via the acoustic matching material.

Then, the procedure for pressing and holding the subject and the configuration necessary for the procedure will be described with reference to FIG. 1B. When the procedure door 2 (not shown) is opened, the holding plate 6 and the pressing plate 7 are seen at the back of the procedure door aperture 3 indicated by the dashed line. In the position relation, the procedure door aperture 3 and the procedure door 2 are provided in the direction (in-plane direction) along one pressing and holding surface of the holding plate 6 and the pressing plate 7, that is, two plates which press and hold the subject. In other words, the aperture and the door are provided in the position in which the plane extending each of the plates crosses the outer cover. In the state that the procedure door is closed, the light is not emitted to the outside of the apparatus. Further, the pressing and holding surface is a surface in which each of the plates and the subject are contacted with each other when the subject is pressed and held.

The subject inserted from the subject insertion hole 5 by the subject person enters between the holding plate 6 and the pressing plate 7. Then, the pressing mechanism 8 is operated to move the pressing plate 7, thereby pressing and holding the subject. The pressing and holding method is as follows. First, the subject person inserts the subject into the subject insertion hole 5. Then, the operator opens the procedure door 2 (not shown), and inserts a hand of the operator from the procedure door aperture 3 to pull the subject. Then, while the operator presses the subject on the pressing plate 7 side with a palm of the operator, the pressing mechanism 8 is driven to press the subject. Further, while extending the subject with the palm of the operator, the operator pulls out the hand of the operator to press the subject again. Then, the distance between the holding plate 6 and the pressing plate 7 can be reduced to make the thickness of the subject smaller, whereby the subject can be tightly held.

The PAM apparatus 100 which has been described above in Example 1 can be sealed by providing the outer cover 1 and the procedure door 2, thereby securing the safety of the subject person and the operator from the irradiation light. Further, the procedure door 2 is provided in the direction along the pressing holding surface of the holding plate 6 and the pressing plate 7, so that when the operator inserts the hand of the operator from the procedure aperture door 3 into the apparatus to perform the procedure onto the subject, the holding plate 6 and the pressing plate 7 cannot be an obstacle. Therefore, the subject is pressed and held by pulling or pressingly extending the subject, so that the pressing and holding distance can be minimum. As a result, the irradiation light can reach deep in the subject, so that the region in which the irradiation light cannot reach there can be eliminated or reduced. Further, since the intensity of the light which reaches the inside of the subject can be increased, the sound pressure of the photoacoustic wave can also be increased, whereby the contrast resolution of acquired image data can be improved.

In the above description, the method for pressing and holding the subject of the subject person who lies on her stomach on the bed 4 has been described. The configuration for pressing and holding the subject is not limited to this. For instance, as shown in FIG. 1C, the configuration in which the subject person stands, as in the typical X-ray mammography, and inserts her breast into the subject insertion hole 5 to press and hold the breast can also be taken. Also, in this case, the procedure door 2 is provided to the outer cover 1, and the operator inserts a hand of the operator from the procedure door aperture 3 to draw the breast. Such configuration is also applicable to the PAM apparatus of the following examples.

### Example 2

In Example 1, the procedure door aperture 3 and the procedure door 2 of the outer cover 1 are provided in the direction along the pressing and holding surface of the holding plate 6 and the pressing plate 7, whereby the operator can easily perform the procedure when pressing and holding the subject. In Example 2, in order that the operator can perform the procedure more easily, the subject can be observed.

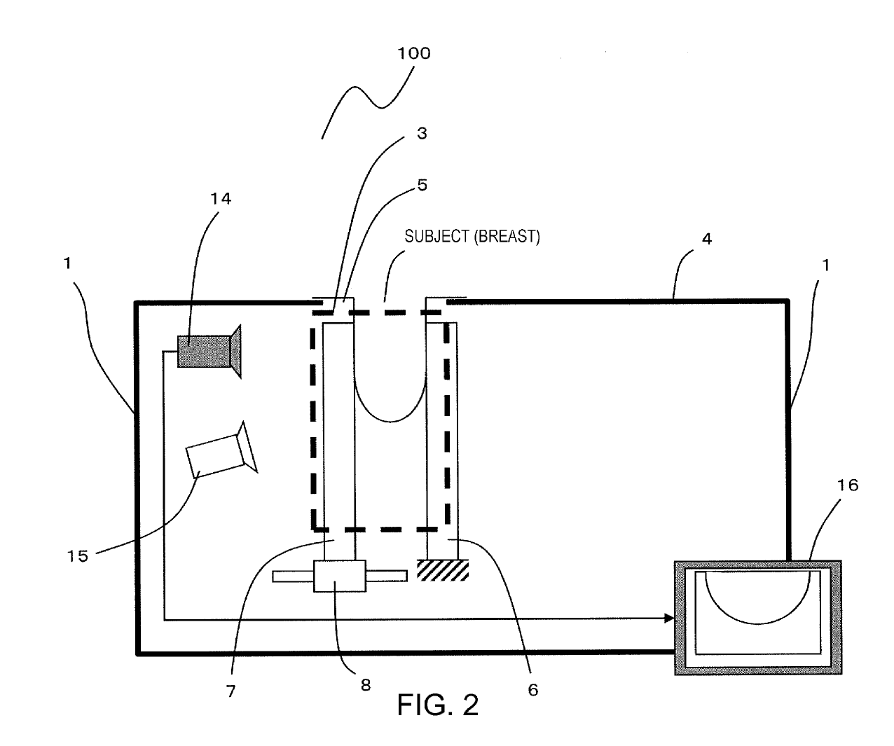

FIG. 2 shows the configuration of the PAM apparatus 100 of the present example. The same components as FIG. 1B are indicated by similar reference numerals and the description will not be repeated. In addition, to simplify the drawing, the laser light source, the optical system, the optical scanning mechanism, the probe, and the probe scanning mechanism are not drawn. As shown, the PAM apparatus of the present example has a camera 14 in the perpendicular direction of the pressing plate 7, and a light 15 which lights the subject. A monitor 16 which displays an image from the camera 14 is provided to the outer cover 1. As the camera 14, for instance, a video camera can be used.

In this example, the camera 14 is provided across the pressing plate 7 on the side in which the subject is imaged. The installation position is not limited to this, and the camera 14 may be provided on the holding plate 6 side. Further, the light 15 is suitably provided in the same direction as the camera 14, which is not limited. For instance, the light 15 may light the subject from below or may light the subject from the opposite side of the camera 14. Furthermore, since it suffices that the monitor 16 can display the pressed and held subject to the operator, the monitor 16 may be provided inside or outside of the PAM apparatus 100. That is, an image imaged by the camera may be output to the monitor installed in the PAM apparatus or may be output to the monitor apart from the apparatus.

The PAM apparatus 100 of the above configuration lights the subject with the light 15, and displays an image showing the pressed and held state of the subject imaged by the camera 14 on the monitor 16. For this reason, the operator can easily observe the subject, and can perform the procedure of pressing and holding the subject more easily.

### Example 3

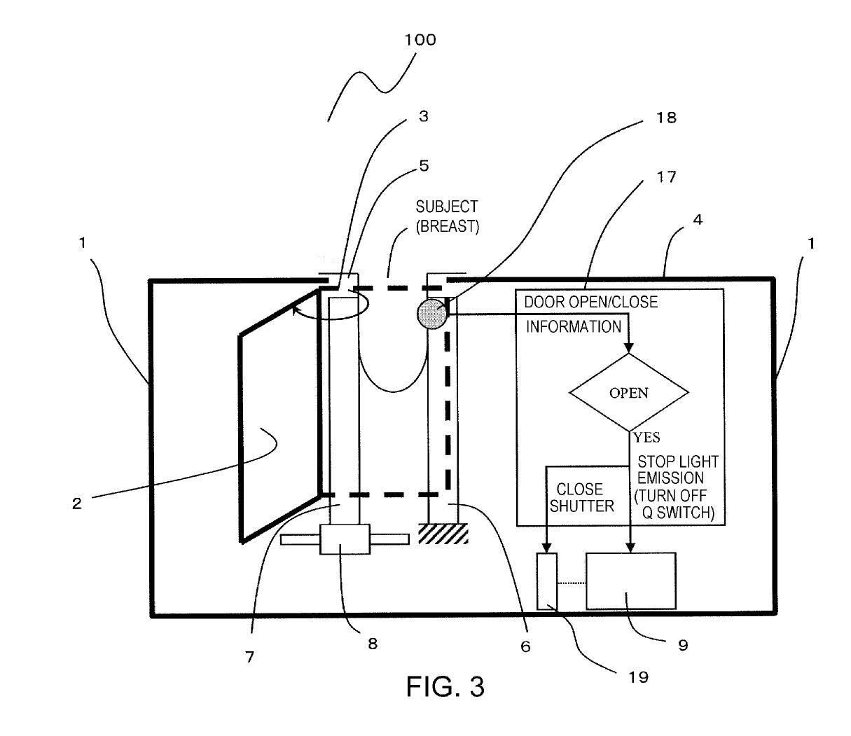

In this example, the emission control of the irradiation light when the door is opened will be described. FIG. 3 shows the inner configuration of the PAM apparatus 100 of this example. The same components as FIGS. 1A and 1B are indicated by similar reference numerals and the description will not be repeated. In addition, to simplify the drawing, the optical system, the optical scanning mechanism, the probe, and the probe scanning mechanism are not drawn. As shown, the PAM apparatus 100 of this example has a controller 17, a door open/close detection sensor 18, and a shutter 19.

To secure the safety of the subject person and the operator, when the procedure door 2 is opened, it is necessary to stop the light emission of the laser light source 9 or to intercept the irradiation light. The control is performed by the controller 17. In addition, the door open/close detection sensor 18 which detects the opening and closing of the procedure door 2 is provided on the procedure door 2 or the procedure door aperture 3. As the door open/close detection sensor 18, a contact type limit sensor is preferable. In addition, the shutter 19 for intercepting the laser light emitted from the laser light source 9 is provided. Further, although the shutter 19 is shown so as to be provided outside the laser light source 9, it may be provided inside the laser light source 9.

Incidentally, to stop the light emission of the laser light source 9, there are a method for turning off a Q switch and a method for turning off a flash lamp. With the method for turning off the Q switch, the laser light source 9 can be held excited, so that it is unnecessary to take long time to restart the laser light source 9 and the light emission can be immediately restarted from the state that the light emission is stopped. In addition, with the method for intercepting the laser by closing the shutter 19 without stopping the light emission, the laser light source 9 can be held excited, so that the light emission can be immediately restarted. Therefore, to stop the light emission, using either of the method for turning off the Q switch and the method for closing the shutter, the light emission can be immediately restarted to quickly acquire the image of the subject.

Accordingly, when the controller 17 turns off the Q switch of the laser light source 9 or closes the shutter 19 when the door open/close detection sensor 18 detects the opening of the procedure door 2. Thereafter, when the door open/close detection sensor 18 detects the closing of the procedure door 2, the controller 17 turns on the Q switch of the laser light source or opens the shutter to restart the light emission. Further, from the viewpoint of improving the safety, when the door open/close detection sensor 18 detects the opening of the procedure door 2, the controller 17 preferably turns off the Q switch of the laser light source 9 and closes the shutter 19.

As described above, in the PAM apparatus of this example, when the opening of the procedure door 2 is detected, the controller stops the laser light emission and intercepts the irradiation light, whereby the safety of the subject person and the operator from the irradiation light can be secured.

### Example 4

In this example, the configuration in which the laser light is intercepted to secure the safety of the subject person and the operator will be described. As in Example 3, the PAM apparatus of this example can perform control which turns off the Q switch to stop the light emission or control which intercepts the irradiation light by the operation of closing the shutter.

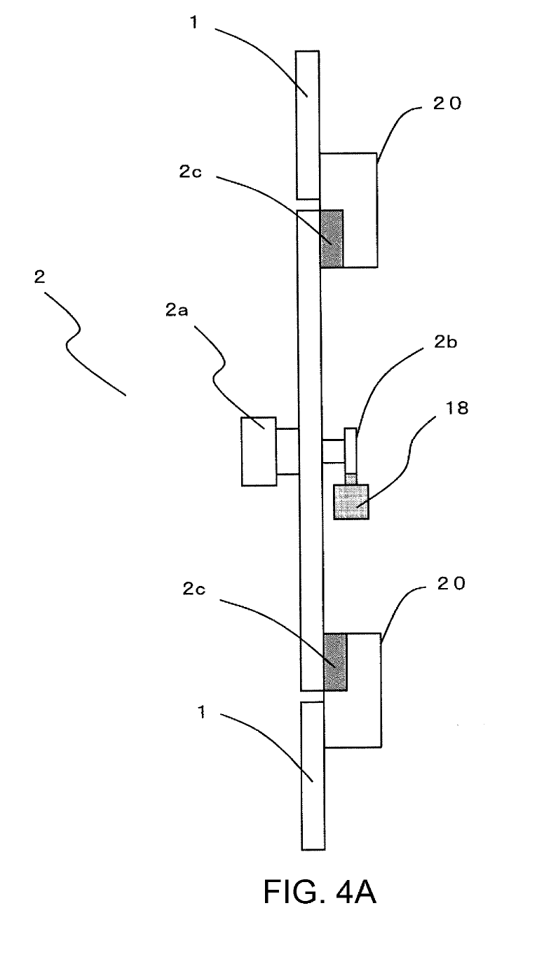

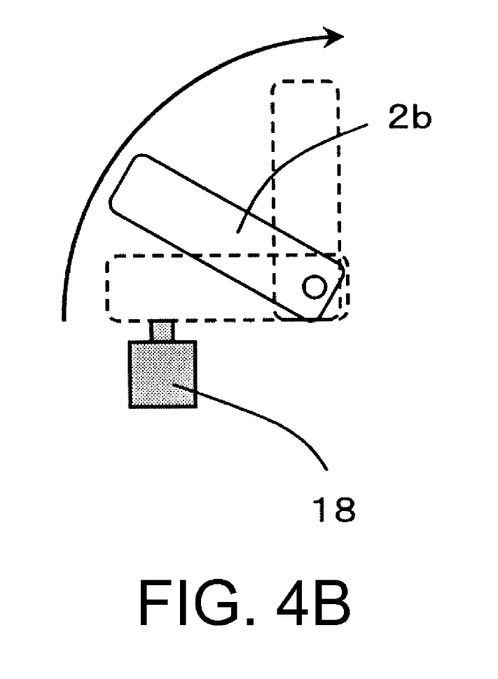

FIG. 4A is a side view of the procedure door 2. The procedure door 2 has a door knob 2a and a door lock 2b for opening and closing the procedure door 2. The door open/close detection sensor 18 is mounted on the door lock 2b so as to react therewith. FIG. 4B shows this state. FIG. 4B shows a rear view centering on the door lock 2b. In addition, as shown in FIG. 4A, in order that the procedure door 2 cannot be immediately opened even when the door lock 2b is unlocked, a magnet catch 2c is provided on a frame 20 of the PAM apparatus 100 body near the aperture of the procedure door 2.

In the PAM apparatus which has been described above in this example, before the procedure door 2 is opened, the door open/close detection sensor 18 can detect the movement of the door lock 2b. Then, even when the door lock 2b is completely rotated in the direction opening the door, the procedure door 2 cannot be easily opened owing to the magnet catch 2c. Therefore, when the door open/close detection sensor detects the movement of the door lock, the Q switch is turned off or the shutter is closed to control the laser light, so that the light irradiation to the subject person and the operator can be prevented and measurement can be safely performed. In addition, the turning-off of the Q shutter and the closing of the shutter are preferably performed together from the viewpoint of improving the safety.

Further, in Examples 3 and 4, to secure the safety of the subject person and the operator, the light emission of the laser light source is stopped or the laser light is intercepted. Alternatively, the procedure door may not be opened during the emission of the laser light. Specifically, the PAM apparatus has an optical sensor, and while the optical sensor detects part of the laser light emitted thereto, the door knob of the procedure door may not be rotated in the state that the procedure door is locked from the inside of the outer cover.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2010-098024, filed on Apr. 21, 2010, which is hereby incorporated by reference herein in its entirety.

1. A subject information acquiring apparatus comprising:
a cover which intercepts light;
two plates which hold a subject;
a probe which receives an acoustic wave generated from the subject to which the light is emitted from a laser light source; and
a cover which covers the plates and the probe and prevents the light is emitted from the laser light source form emitting to the outside of the apparatus,
wherein the cover has a first aperture for inserting the subject as part of a subject person into the cover, and a second aperture which can be opened and closed so that an operator can perform a procedure by inserting a hand of the operator into the cover in the position in which a plane extending each of the plates crosses the cover.

2. The subject information acquiring apparatus according to claim 1, further comprising:
light which lights the subject; and
a camera which images the subject,
wherein the image of the subject imaged by the camera is output to a monitor.

3. The subject information acquiring apparatus according to claim 1, further comprising:
a sensor which detects the open/close state of the second aperture;
a shutter which intercepts the light from the laser light source; and
a controller which performs at least either one of control for turning off a Q switch of the laser light source and control for intercepting the light from the laser light source by the shutter when the sensor detects the opening of the second aperture.

4. The subject information acquiring apparatus according to claim 3,
wherein the second aperture is a door having a door lock,
wherein the sensor detects the opening of the second aperture when the door lock is moved from the position in which the door is locked.

5. The subject information acquiring apparatus according to claim 4, wherein the cover has a magnet catch which holds the door.
